(19) United States
(12) Patent Application Publication
(10) Pub. No.: US 2002/0128679 A1
(43) Pub. Date: Sep. 12, 2002

(54) Cerebral protection during carotid endarterectomy and methods of use

(75) Inventors: Roman Turovskiy, San Francisco, CA (US); Jobert Balceta, San Jose, CA (US); Ross S. Tsugita, Mountain View, CA (US); Peter Thornton, Los Altos, CA (US); Brian P. Nuel, Cupertino, CA (US); Lorraine M. Martinez, Fremont, CA (US)

Correspondence Address: LYON & LYON LLP, 633 WEST FIFTH STREET, SUITE 4700, LOS ANGELES, CA, 90071, US

(73) Assignee: EMBOL-X, Inc.

(21) Appl. No.: 09/803,745

(22) Filed: Mar. 8, 2001

(51) Int. Cl. A61M 29/00

(52) U.S. Cl. 606/200

(57) ABSTRACT

A shunt and method of use for maintaining distal blood flow during an arteriotomy procedure is disclosed. The shunt includes first and second tubular members having proximal ports, distal ports, and lumens therebetween. The distal port of the second tubular member is adapted for releasable attachment to the proximal port of the first tubular member. A second lumen merges and communicates at its distal end with the lumen of the first tubular member and includes a hemostatic valve attached to its proximal end. In using the apparatus for performing open endarterectomy, a filter device is inserted into the vessel and deployed downstream the region of interest in the internal carotid artery. The distal end of the shunt is advanced over the filter device and secured onto the artery. The proximal end of the shunt is inserted upstream the region of interest, typically in the common carotid artery.

### FIELD OF THE INVENTION

The present invention relates to open carotid endarterectomy. More particularly, it relates to methods and apparatus for improving endarterecomy procedures by using blood filtration to protect the patient from embolization and vascular shunting to maintain blood perfusion during these vascular surgeries.

### BACKGROUND OF THE INVENTION

Arteriosclerosis, generally known for thickening and hardening of the arterial wall, is responsible for the majority of deaths in the United States and most westernized countries. Atherosclerosis, one type of arteriosclerosis, is the cause for disorder of the larger arteries that underlies most coronary artery disease, aortic aneurysm, arterial disease of the lower extremities, and cerebrovascular disease. Atherosclerosis is characterized by an accumulation of lipid-filled smooth muscle cells, macrophages, and fibrous tissues, commonly known as atheroma or plaque, in focal areas of cardiovascular tissues, especially the carotid arteries. The atheromatous lesions in the extracranial carotid vessels, i.e., the common carotid arteries (CCA), the internal carotid arteries (ICA), and the external carotid arteries (ECA), progress through a stage in which brain blood flow is marginal to the stage of occlusion, which results in insufficient cerebral perfusion, with brain death and clinical stroke. Therefore, it is advantageous to prophylactically treat the hemodynamically significant carotid lesions (i.e., greater than 80 percent occlusion of the arterial lumen) to prevent stroke.

Endarterectomy is a surgical procedure which generally includes the removal of diseased intimal lining of an artery and is most commonly used to treat vascular insufficiency of the carotid, femoral, and popliteal arteries. In a typical carotid endarterectomy, the surgery is performed with the patient under general anaethesia with the head extended and turned to the side opposite the diseased carotid artery. The surgeon makes an incision in the cervical skin crease, centering on the anterior border of the sternocleidomastoid muscle. He then opens the fascia over the internal jugular vein and divides the common facial vein, which is disposed over the carotid artery. Anticoagulant, such as heparin, is infused intravenously to prevent clotting in any stagnant areas of the vessels. The CCA, ICA, and ECA are dissected at the levels not involved in the disease process and are then occluded by clamping. Blood pressure is measured in the ICA before and after applying the vascular clamps to the CCA and the ECA. Measurement of this carotid “stump pressure” is necessary to assess the adequacy of collateral circulation to the brain. Alternatively, TCD, blood velocity, LOC, decreased cognition, EEG or other methods could be used to determine whether or not a shunt is needed. Having the stump pressure above 50 mm Hg suggests adequacy of collateral circulation to support cerebral metabolism during the endarterectomy, and indicates that a temporary shunt is unnecessary. If the stump pressure is lower than 50 mm Hg, brain perfusion during carotid occlusion may be inadequate, thereby requiring a shunt to bypass the clamped region of the artery to maintain cerebral perfusion.

Since the usual site of the internal carotid artery disease is at and just distal to its origin at the bifurcation of the CCA, an incision is made in the anterolateral aspect of the CCA to a point beyond the plaque. The diseased intima and the media-adventitia of the artery are separated, and the atheromatous material is removed, first from the CCA, then from the ECA, and generally last from the ICA. The artery is then carefully reconstructed and may require a vein patch if there is insufficient tissue for construction. Arteriograms are then obtained to assess the re-established vascular patency.

The above-described procedure, however, suffers from a deficiency which relates to the escape of embolic material which may lead to devastating neurologic complications, particularly when emboli pass through the internal carotid artery. Emboli may be produced through any step of the procedure where mechanical forces are applied to the artery, and these manipulations include clamping, unclamping, applying a tourniquet, dissecting the vessel, inserting and removing a bypass shunt, removing atheromatous material, cleaning the affected site, and suturing the vessel. Therefore, a need exists for an improved endarterectomy procedure and apparatus that will enable the surgeon to minimize the production of embolic material and to prevent the escape of embolic material during carotid endarterectomy, arterotomy, and other vascular surgeries

### SUMMARY OF THE INVENTION

A dramatic improvement in the neurologic outcome of patients undergoing carotid endarterectomy, and arteriotomy procedures generally, can be achieved by using a blood filter device to capture and remove dislodged embolic material and a shunt to maintain vascular perfusion during the surgical procedure in accordance with our invention. Thus, the invention provides novel methods and apparatus for protecting a patient from embolization during arteriotomy procedures. In one embodiment, the invention provides a bypass tubing or indwelling shunt, having a main lumen for blood bypass and a second, branching lumen adapted to receive an elongated blood filter and to allow passage of same into an artery distal to the endarterectomy region. The branching secondary lumen can either merge and communicate with the main lumen of the shunt, or may extend to a distal opening separate from the blood bypass lumen of the device. A hemostatic valve is included in the proximal end of the second lumen to prevent blood loss from the shunt.

The blood filter device typically includes a catheter sheath, an elongated control member, a control mechanism at a proximal end of the control member, and a filtration assembly which includes an expandable filter, typically comprising an expansion frame and filter mesh at a distal region of the control member, the expansion frame being operable to enlarge from a contracted condition to an expanded condition which covers all of, or a substantial portion of the cross-sectional area of a vessel. In alternative embodiments, a filter is disposed on a guidewire or tubing for use in carotid artery bypass to capture clots and atherosclerotic material released during endarterectomy.

In another embodiment, the shunt comprises two tubular members. The first tubular member has a lumen that communicates with a distal port and first and second proximal ports. The second member has a lumen, which communicates with a proximal port and a distal end, which is adapted for releasable attachment to the first proximal port of the first tubular member. A filter device comprising an elongate member is insertable through the hemostatic valve included in the second proximal port of the first tubular member. In certain embodiments, the first and/or second tubular member includes a valve for regulating blood flow through the shunt, thereby maintaining optimal vascular perfusion. A manometer may be included in the distal end of the first tubular member to monitor blood pressure downstream the atheromatous lesion. This apparatus is especially useful in performing endarterectomy in patients having tenuous cerebral perfusion pressure (i.e., having ICA pressure slightly above 50 mmHg) and initially not requiring a shunt. The second tubular member can easily be connected to the first tubular member intra-operatively when unexpected cerebral hypoperfusion occurs, e.g., as in systemic hypotension or cardiogenic shock, to maintain blood flow to the brain.

In still another embodiment, the shunt comprises a tubular member having a perfusion lumen that communicates with a proximal end and a distal end. An expandable balloon occluder which communicates with an inflation lumen and port is mounted on the proximal end and/or the distal end of the shunt. In certain embodiments, expandable occlusion membrane(s) are mounted on the proximal and/or distal ends of the shunt. The expanded balloon(s) or membrane(s) are capable of occluding the vascular lumens to seal the shunt against the vessel, thereby replacing vascular clamps. An expandable filter is mounted on the distal end of the shunt, proximal to the occluding balloon. The filter is contracted or expanded by advancing a slideable sheath covering the shunt. In certain embodiments, the shunt also includes a second, branching lumen adapted for infusion of fluid, such as saline or lactated Ringer, and for aspiration. The branching secondary lumen typically extends to a distal opening separate from the blood bypass lumen of the shunt.

According to the methods of the present invention, an affected region of an artery is isolated, clamped, and dissected as disclosed in Loftus, Carotid Endarterectomy Principles and Techniques; Quality Medical Publishing, Inc.; St. Louis, Mo., 1995, and Smith, The Surgical Treatment of Peripheral Vascular Disease, Chapter 142, in “The Heart, Arteries, and Veins,” Vol. 2, Ed. J. Willis Hurst; McGraw-Hill Information Services Corp., 1990, both incorporated herein by reference in their entirety. A shunt having a balloon occluder and filter mounted at its distal end as described herein is then inserted so that the proximal end and the distal end are positioned, respectively, upstream and downstream of the atheromatous lesion. The blood filter is released and expanded by pulling the sheath proximally, and the balloon occluders mounted on the proximal and distal ends of the shunt are expanded by infusion of air or saline through the inflation lumen(s). After endarterectomy is performed to remove atherosclerotic material from the affected region of the artery, the balloon occluders are deflated and the filter is collapsed. The shunt and the captured embolic debris in the filter are removed from the artery.

In another method for performing open surgical endarterectomy, a filter device having an elongate member is first inserted downstream of the atheromatous lesion and the filter is expanded to cover a substantial cross-sectional area of the artery. The distal end of a shunt is then loaded onto the elongate member and advanced over the elongate member of the filter device to position within the ICA. The distal end of the shunt is then secured by a distal artery clamp, while the proximal region of the shunt is inserted into the proximal artery, i.e., the CCA or ICA, and is secured by a clamp proximal to the region of arteriotomy. After endarterectomy is performed to remove the occluding lesion, the shunt is removed and the incision on the artery is closed. The filter is collapsed and removed with the captured embolic debris.

In another method using the shunt having two releasably attached tubular members as disclosed herein, an incision is made proximal to the site where the common carotid artery cross-clamp will be placed. The filter device, in a contracted state having the sheath over the filter, is inserted through the incision distal to the region of arteriotomy. The distal end of the first tubular member is then advanced over the wire or elongate member of the filter device to position downstream the atheromatous lesion. The filter is then expanded by pulling the sheath proximally, the filter expanding to cover a substantial cross-sectional area of the artery. The proximal end of the second tubular member is inserted upstream the atheromatous lesion, typically in the CCA. The common and external carotid arteries are then clamped. The distal end of the second tubular member and the proximal end of the first tubular members are joined to maintain cerebral perfusion, if the blood pressure in the ICA distal to the clamping is inadequate. The ICA is then clamped and incised, plaque removed, the operative site rinsed with sterile saline or water, and the shunt removed from the common carotid artery. The proximal and distal cross-clamps are removed, and circulation through the repaired carotid artery is restored as discussed herein. The carotid artery, with or without a graft, is closed by suturing from both ends of the incision inward. The filter, including captured embolic material, is collapsed by advancing the sheath over the filter and is retracted after several minutes, typically at least 5 minutes, more preferably at least 10 minutes. The re-established lumenal patency of the artery can be assessed by injecting radiopaque material into the artery to be visualized under fluoroscopy.

It will be understood that there are several advantages to using the apparatus and methods disclosed herein for performing open endarterectomy, especially the on the extracranial carotid arteries. For example, the apparatus (1) provides a filter device to capture embolic debris generated during the procedures, thereby minimizing the risk of perioperative stroke, (2) provides a shunt for maintaining cerebral perfusion, thereby further reducing the risk of perioperative morbidity, (3) provides balloon occluders as an alternative to using vascular clamps which commonly cause injuries to vessel, such as hematoma or dissection, (4) provides a filter device which can be used in conjunction with a standard single-lumen indwelling shunt, and (5) can be used in performing endarterectomy on various arteries, including the aorta, the iliac, the femoral, and the popliteal arteries.

### BRIEF DESCRIPTION OF THE DRAWINGS

FIG. 1A depicts a filter device introduced into a carotid artery through an introducer upstream an atheromatous lesion.

FIG. 1B depicts the filter device of FIG. 1A inserted downstream the atheromatous lesion.

FIG. 1C depicts a single-lumen shunt used in conjunction with the filter device of FIG. 1B.

FIG. 2A depicts an expanded filter insertable through another embodiment of the introducer.

FIG. 2B depicts the filter device of FIG. 2A collapsed and retracted within the introducer.

FIG. 2C depicts the filter/introducer assembly of FIG. 2A inserted through an arteriotomy incision.

FIG. 2D depicts the filter/introducer assembly of FIG. 2A inserted through an incision prior to arteriotomy.

FIG. 3 depicts a shunt having a filter included in its distal end.

FIG. 4A depicts an embodiment of a shunt/introducer assembly.

FIG. 4B depicts another embodiment of a shunt/introducer assembly having a detachable shunt inserted downstream an atheromatous lesion.

FIG. 4C depicts the shunt/introducer assembly inserted downstream the atheromatous lesion.

FIG. 4D depicts the shunt of FIG. 4C inserted downstream of the filter.

FIG. 5A depicts an embodiment of a filter in its resting contracted state.

FIG. 5B depicts the filter of FIG. 5A expanded by pulling its elongate member.

FIG. 5C depicts another embodiment of a filter in its resting expanded state.

FIG. 5D depicts the filter of FIG. 5C contracted by pulling its actuator tube.

FIG. 6A depicts an embodiment of integrated filter and shunt.

FIG. 6B depicts the filter of FIG. 6A expanded within a distal region of the shunt.

FIG. 7A depicts another embodiment of an integrated filter and shunt having a plurality of perfusion ports.

FIG. 7B depicts the filter of FIG. 7A expanded within a distal region of the shunt.

FIG. 8A depicts another embodiment of an integrated filter and shunt.

FIG. 8B depicts the filter of FIG. 8A expanded distal the shunt.

FIG. 9A depicts the distal end of a shunt inserted over a filter sheath.

FIG. 9B depicts the filter and sheath inserted in the internal carotid artery for performing endarterectomy.

FIG. 10A depicts the shunt of FIG. 9A inserted over another embodiment of a filter deployed in the internal carotid artery.

FIG. 10B depicts the filter of FIG. 10A contracted by a sheath.

FIG. 10C depicts the expanded state of the filter of FIG. 10B.

FIG. 11A depicts using a balloon occluder to isolate blood flow downstream an atheromatous lesion during endarterectomy.

FIG. 11B depicts the filter device of FIG. 11B deployed downstream the shunt and occluder of FIG. 11A.

FIG. 12A depicts a balloon occluder isolating blood flow upstream an atheromatous lesion during endarterectomy.

FIG. 12B depicts the shunt of FIG. 12A inserted downstream the filter.

FIG. 13A depicts an expanded balloon occluder disposed about a distal region of the shunt.

FIG. 13B depicts the contracted state of the balloon occluder of FIG. 13A.

FIG. 14 depicts balloon occluders disposed about the proximal and distal regions of a shunt.

FIG. 15 depicts another-embodiment of the introducer, which includes lumens for blood flow, aspiration, insertion of filter/sheath, and balloon inflation.

FIG. 16 depicts another embodiment of the shunt having lumens adapted for insertion of filter and balloon occluder.

FIG. 17A depicts another embodiment of the shunt having a filter and balloon occluder mounted at its distal region.

FIG. 17B depicts the filter of FIG. 17A expanded by withdrawing a sheath.

FIG. 18 depicts another embodiments of the shunt, which includes a valve for controlling blood flow.

FIG. 19 depicts another embodiment of the shunt having balloon occluders mounted at its proximal and distal ends, communicating with separate inflation lumens.

FIG. 20A depicts another embodiment of a split-shunt device with deployed filter.

FIG. 20B depicts the distal end of the shunt of FIG. 20A deployed within a vessel.

FIG. 20C depicts the distal and proximal ends of the shunt of FIG. 20A deployed within a vessel.

FIG. 20D depicts the removal of the distal end of the shunt of FIG. 20A from the vessel.

### DETAILED DESCRIPTION

The devices and methods disclosed herein function to prevent embolic material from migrating downstream (into the brain, the kidneys, the lower extremities, etc.) during vascular surgery. The devices and methods herein are useful during any procedure where vessels are incised for the purpose of removing occlusions or performing other types of repair that may require the use of shunting to maintain distal blood flow.

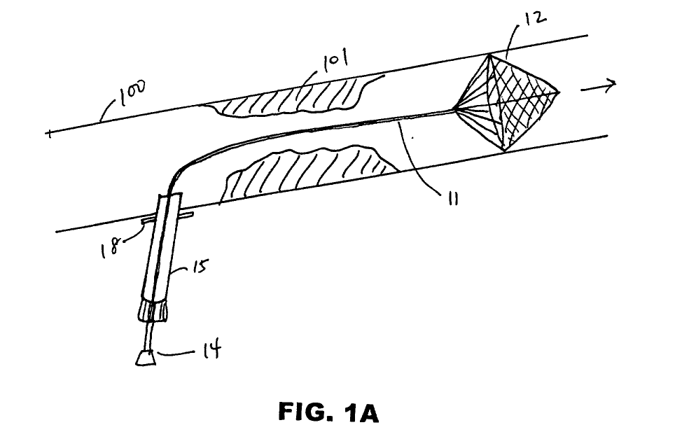

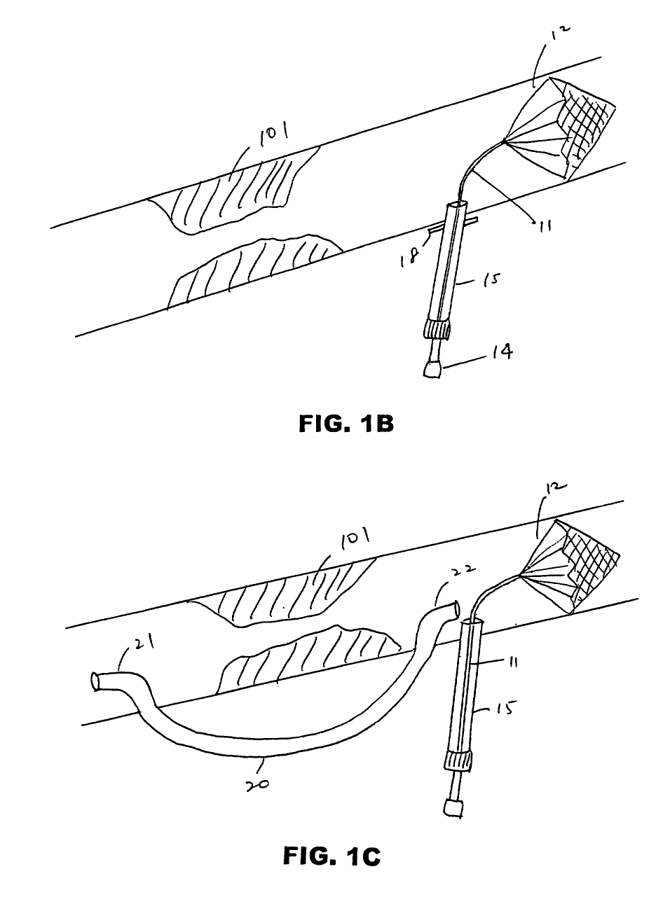

FIG. 1A depicts an embodiment of a filter device deployed in carotid artery 100 proximal to atheromatous lesion 101. The device comprises filter assembly 12 mounted on elongate member 11 (guidewire, sheath, etc.). Elongate member 11 is insertable in the lumen of introducer 15 and is connected to filter delivery cartridge 14 at its proximal end. Suture flange 18 is mounted on a distal region of introducer 18.

The device of FIG. 1A is useful in performing endarterectomy in patients with adequate cerebral perfusion and not requiring a shunt. Introducer 15 is first inserted into the lumen of carotid artery 100 after an incision is made upstream atheromatous lesion 101. Alternatively, the filter device of FIG. 1A can be inserted downstream atheromatous lesion 101 as depicted in FIG. 1B. Sutures are placed on flange 18 to secure introducer 15 onto the vessel wall. The filter device, having filter assembly 12 placed in a contracted state, is inserted through the lumen of introducer 15 and advanced downstream of lesion 101. Once in place, filter delivery cartridge 14 is operated to expand filter 12 so that it covers most, if not all, of the cross-sectional area of vessel 100. Vascular clamps are then placed on the vessel upstream and downstream the atheromatous lesion, the clamps being placed upstream of the developed filter. After the surgeon performs arteriotomy and endarterectomy to remove lesion 101, filter assembly 12 is collapsed. The captured embolic material, such as calcium, plaque, thrombi, and tissue debris, generated during the procedure are then removed with the collapsed filter, thereby preventing distal embolization.

In performing endarterectomy on patients requiring a shunt, the filter device described in FIG. 1B can be used in conjunction with standard single-lumen indwelling shunt 20 as depicted in FIG. 1C. Shunt 20 is generally inserted in the artery proximal and distal atheromatous lesion 101 after arteriotomy. Vascular clamps are placed over proximal end 21 and distal end 22 of the shunt during endarterectomy, and the shunt maintains blood flow to the brain.

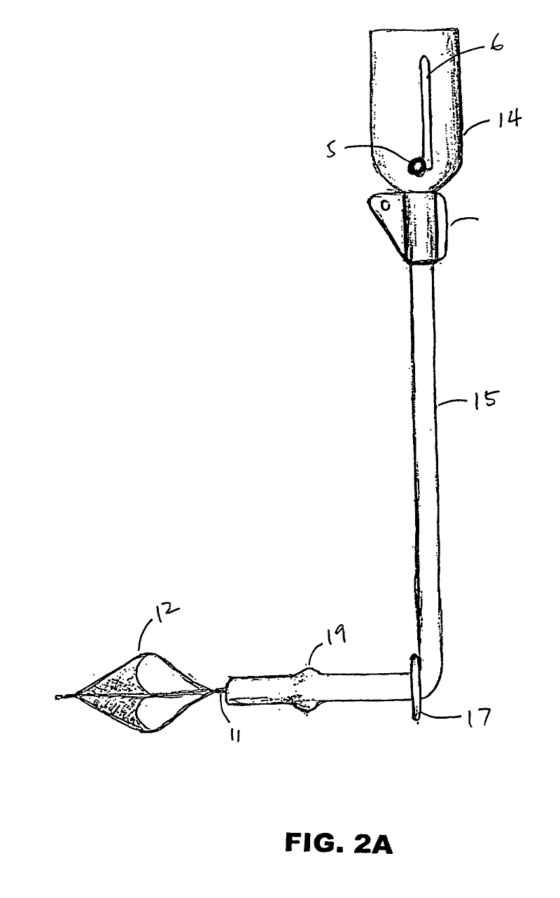

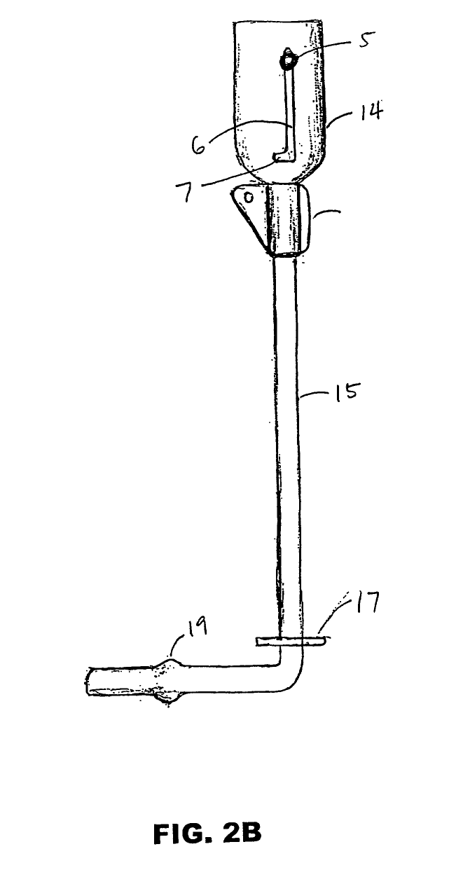

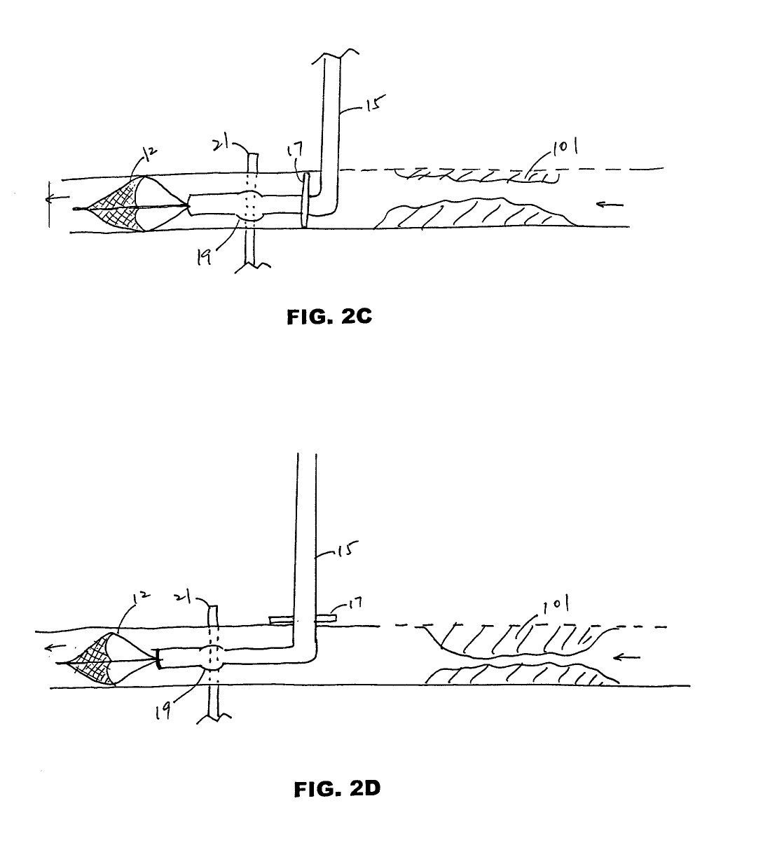

Another filter/introducer apparatus adapted for use in open surgical carotid endarterectomy is depicted in FIG. 2A. Filter assembly 12 is mounted on the distal end of elongate member 11 and is operable from the proximal end of the elongate member which is attached to mechanism 5 included in filter delivery cartridge 14. The filter is collapsed and retracted into the lumen of introducer when mechanism 5 slides proximally in slot 6 as depicted in FIG. 2B. After the introducer is inserted in the artery, filter assembly 12 is deployed by sliding mechanism 5 distally in slot 6 until it locks in groove 7, thereby fixing the filter in an open state as depicted in FIG. 2A. The distal region of introducer 15 also includes circumferentially enlarged region 19 for placement of a Javid clamp, thereby fixing the introducer within the vessel, minimizing displacement between the introducer and the vessel, and reducing trauma to the vessel. The distal region of introducer 15 is angled relative to its proximal end to facilitate insertion into an artery. Stopper 17 is sliceable mounted in the distal region of introducer 15 and can be positioned perpendicular to the longitudinal axis of the filter as depicted in FIG. 2A or parallel to the axis as depicted in FIG. 2B.

In use, introducer 15, having filter 12 in a collapsed state, is inserted downstream atheromatous lesion 101 after arteriotomy (shown in broken line) as depicted in FIG. 2C. Filter 12 is expanded. Stopper 17, positioned perpendicular to the longitudinal axis of the filter, minimizes displacement of the introducer and filter in the artery. Clamp 21 is placed over region 19 and endarterectomy is performed with or without a shunt. Alternatively, introducer 15 is inserted through an incision downstream lesion 101 prior to arteriotomy as depicted in FIG. 2D. Stopper 17 is positioned parallel to the longitudinal axis of the filter, thereby stabilizing the introducer on the vessel. After endarterectomy, the filter is collapsed and removed with the captured emboli generated during the procedure.

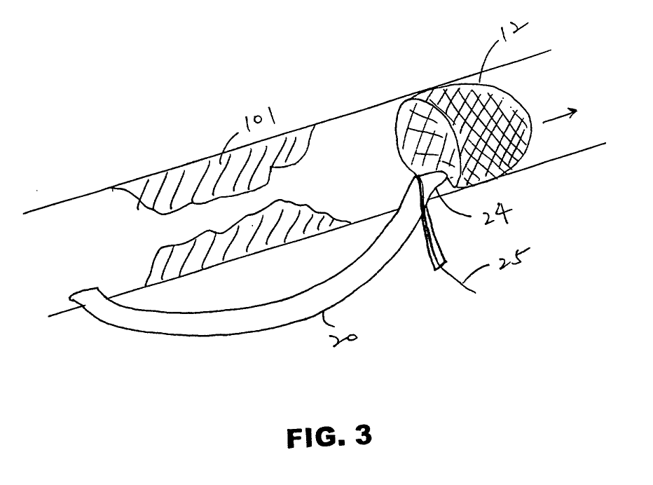

FIG. 3 depicts filter 12 mounted on distal end 24 of shunt 20. The filter is expanded in its resting state and is contracted by pulling on filter collapsing string 25. In use, distal end 24 of shunt 20, having filter 12 in a contracted state, is inserted downstream atheromatous lesion 101. Filter 12 is expanded to substantially cover the perimeter of the vessel wall by releasing string 25 and returning the filter to its expanded resting state. The proximal end of shunt 20 is inserted upstream lesion 101. After endarterectomy, the proximal end of shunt 20 is removed, the arteriotomy is closed, filter 12 is collapsed by pulling on string 25, and distal end 24 of shunt 20 and filter 12 are removed.

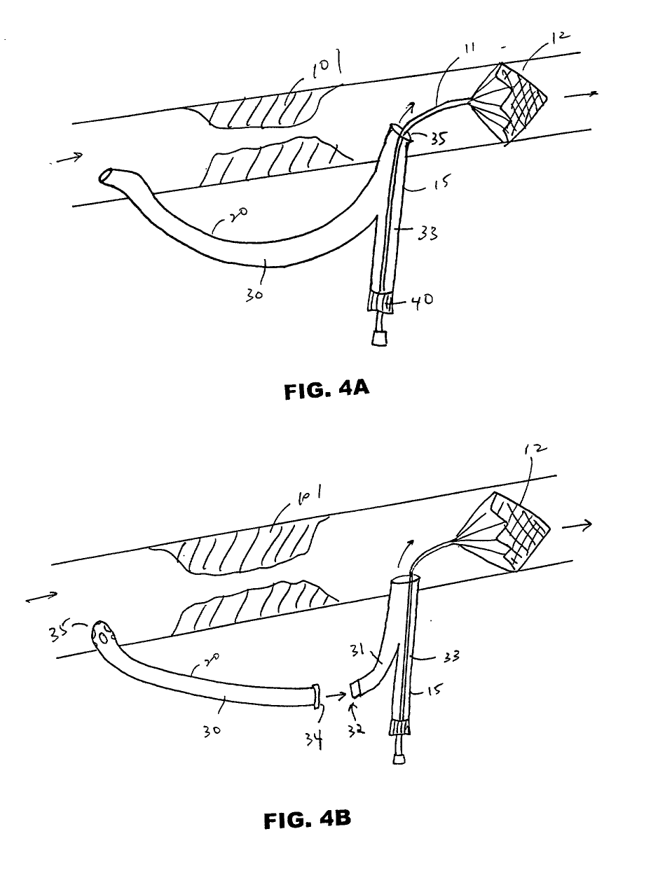

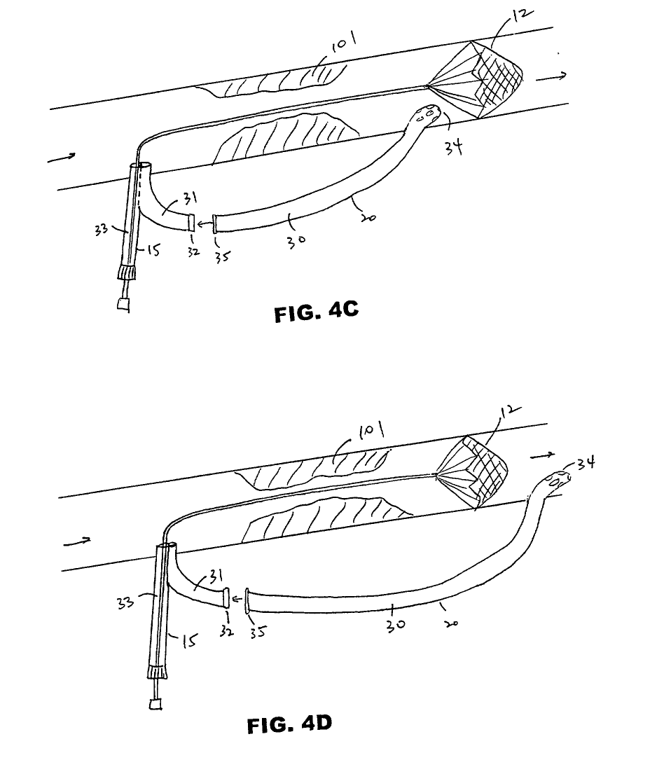

FIG. 4A depicts a shunt/introducer assembly having lumen 30 adapted for perfusion of blood and lumen 33 adapted for insertion of a filter device. Lumens 30 of shunt 20, and lumen 33 of introducer 15 merge and communicate at distal port 35. In use, the collapsed filter on a wire or catheter is inserted and deployed downstream atheromatous lesion 101, and the distal end of the shunt is inserted over the filter wire or catheter into the artery. Filter 12 captures embolic debris. Back-bleeding through the shunt occurs from the distal opening 13 of lumen 15 in order to purge air from within the shunt. After the shunt is purged, the proximal opening of shunt 20 is inserted upstream of lesion 101 and secured by a clamp (not shown). Blood flows from the proximal end to the distal end of the shunt during endarterectomy to maintain vascular perfusion. Hemostatic valve 40, included in the proximal end of lumen 33, prevents backflow of blood.

In another embodiment, shunt 20 is detachable from introducer 15 as depicted in FIG. 4B. Introducer 15 has lumen 31 that communicates with proximal end 32 and lumen 33 that includes hemostatic valve 40 at its proximal end. Shunt 20 has lumen 30 which communicates with distal end 34 and one or more ports in proximal end 35. Distal end 34 is attachable to proximal end 32 of the introducer. In use, the distal end of introducer 15 is inserted downstream of lesion 101. Filter 12 is advanced through the introducer into the artery, and is expanded to capture embolic debris. Proximal end 35 of shunt 20 is inserted upstream lesion 101 and distal end 34 of the shunt is attached to proximal end 32 of the introducer when shunting is required to maintain cerebral perfusion. After endarterectomy, ends 34 and 32 are disconnected, shunt 20 is removed, filter 12 containing captured debris is contracted, and introducer 15 and the filter are removed from the vessel.

Alternatively, the filter introducer described in FIG. 4B is inserted upstream of lesion 101 as depicted in FIGS. 4C and 4D. In use, filter 12 is inserted through introducer 15 upstream of lesion 101 and advanced to a position downstream of lesion 101. Filter 12 is expanded to capture vascular debris. If a shunt is required, proximal end 35 of shunt 20 is attached to proximal end 32 of introducer 20, and distal 34 of the shunt is inserted downstream of lesion 101 as depicted in FIG. 4C. Alternatively, distal end 34 of the shunt is inserted downstream of filter 12 as depicted in FIG. 4D. The detachable shunt/introducer assembly is particularly useful in circumstances where cerebral hypoperfusion occurs intra-operatively, but was not anticipated because the patient appeared to have adequate carotid blood pressure.

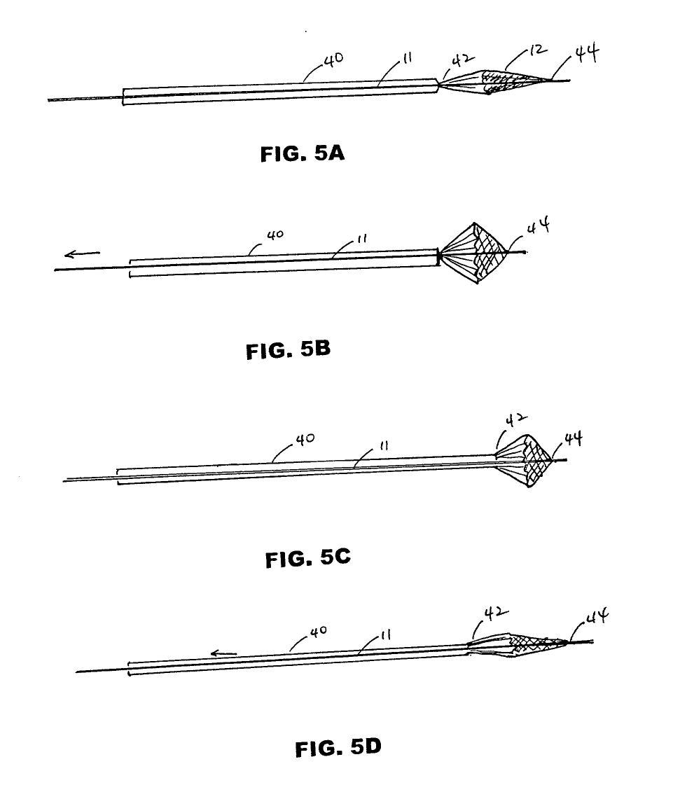

FIGS. 5A through 5D depict filter devices adapted for insertion through an introducer or a shunt. In FIG. SA, the filter device comprises an elongate member 11, e.g., a wire, having filter 12 mounted on its distal end. Elongate member 11 is insertable through actuator tube 40, e.g., a sheath or catheter. Distal end 44 of filter 12 is fixed on elongate member 11 whereas its proximal end 42 is slideable along the elongate member. In its resting state, filter 12 is closed. To expand the filter, the proximal end of elongate member 11 is pulled proximally so that proximal end 42 of the filter abuts the distal end of actuator tube 40, causing filter 12 to buckle outward, thereby expanding the filter as depicted in FIG. 5B.

According to FIG. 5C, distal end 44 of filter 12 is fixed on elongate member 11. Proximal end 42 of the filter is connected to the distal end of actuator tube 40, such that the filter is expanded in its resting state. To insert a contracted filter, actuator tube 40 is pulled proximally against elongate member 11 as depicted in 5D. When the filter is positioned in the region of interest, the actuator tube is released to return the filter to its resting expanded state.

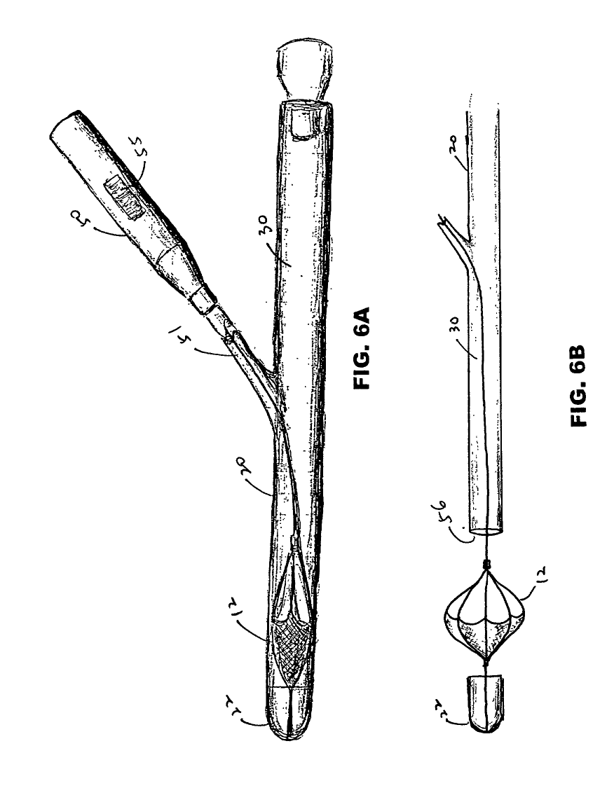

Another embodiment of the integrated filter and shunt assembly where the distal tip of the shunt and filter can be concomitantly inserted and advanced into an artery is depicted in FIGS. 6A and 6B. Prior to insertion, filter 12 is collapsed and retracted within lumen 30 of shunt 20 as shown in FIG. 6A. Elongate member 11 of filter 12 is connected to actuating mechanism 55 at the proximal end of handle 50. Lumen 30 of the shunt communicates with lumen 51 of the handle. Distal end 22 of shunt 20 comprises a rounded tip, adapted to reduce trauma to the vascular wall during insertion. Distal end 22 is releasably attached to port 56 of the shunt, which communicates with lumen 30. Filter 12 is expanded by moving the filter distally, and it opens proximal to distal end 22 as depicted in FIG. 6B.

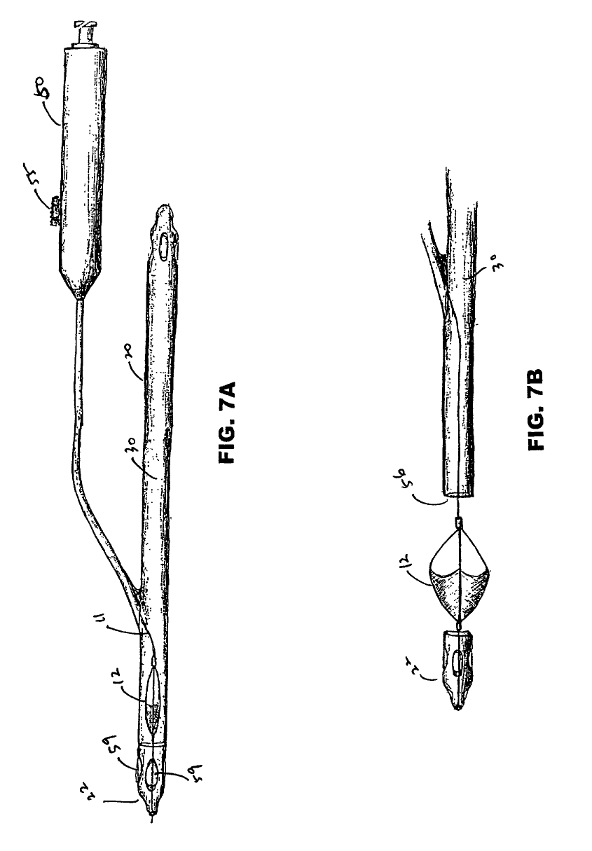

FIG. 7A depicts another embodiment of the filter and shunt assembly useful in patients requiring a shunt during endarterectomy. Filter 12 is closed and retracted within lumen 30 of shunt 20. Distal end 22 of the shunt includes a plurality of infusion ports 59 to facilitate laminar flow in the vessel. Filter 12 is expanded between distal end 22 and port 56 by operating actuating mechanism 55 on handle 50 as depicted in FIG. 7B.

In using the devices described in FIGS. 6A and 7A, distal end 22 of the shunt is inserted into an artery downstream an atheromatous lesion. Actuating mechanism 55 is then operated distally to expand filter 12 to capture embolic debris. The artery is perfused from blood flowing through lumen 30 and port 56 of shunt 20. After endarterectomy, filter 12 is collapsed, retracted into lumen 30 of the shunt, and removed with the captured debris.

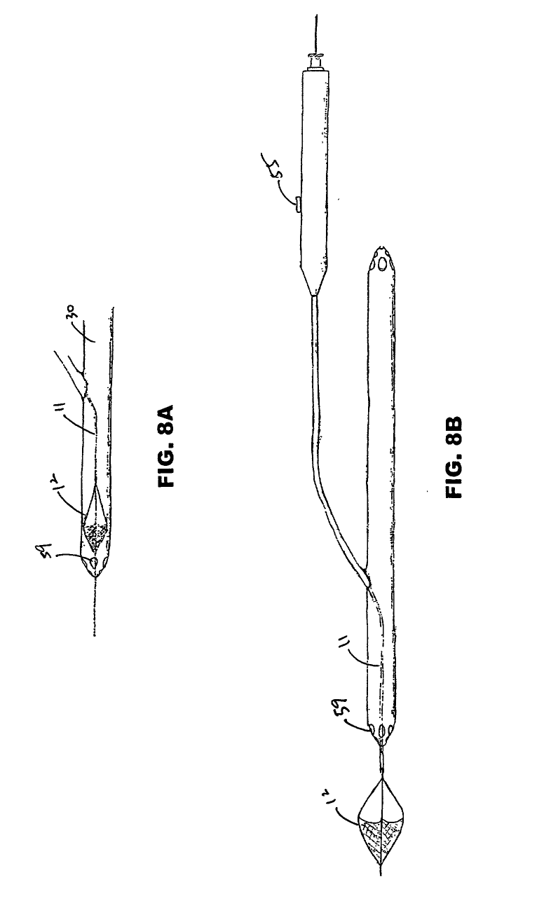

The filter and shunt assembly shown in FIG. 8A differs from the devices in FIG. 7A in that the distal region of the shunt in FIG. 8A is contiguous with its proximal end and is not separable as depicted in FIG. 7A. In using the assembly of FIG. 8A, the distal end of the shunt, having filter 12 contracted within lumen 30, is inserted as a unit into an artery downstream an atheromatous lesion. Filter 12 is then advanced distally and expanded and deployed downstream infusion ports 59 by operating actuating mechanism 55 at the proximal end of elongate member 11. After completion of endarterectomy, the captured embolic debris are secured by the collapsed filter 12 and removed with the filter and shunt.

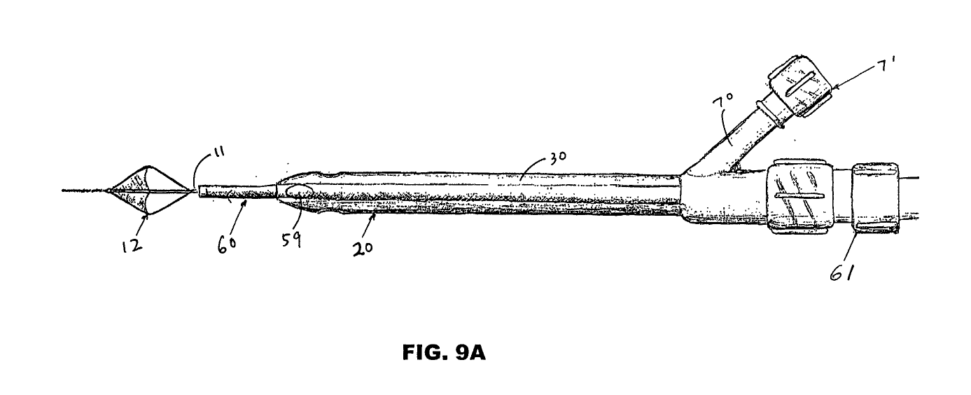

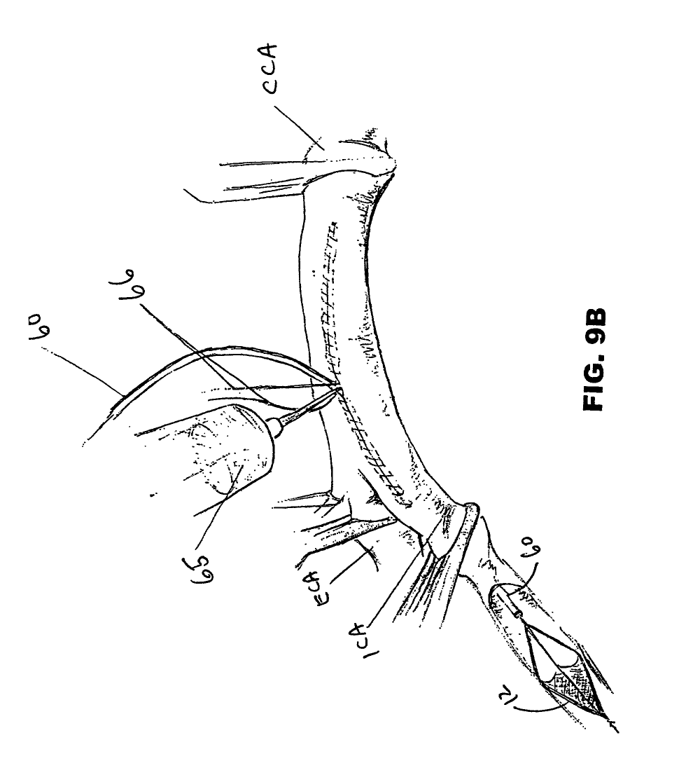

With reference to FIGS. 9A and 9B, the use of a shunt and filter as disclosed herein will be described in the context of an endarterectomy procedure. A typical site of atherosclerotic plaque build-up is in the common carotid artery near the bifurcation of the internal and external carotid arteries. The surgeon generally makes an incision in the neck to expose the segment of carotid arteries having plaque build-up. A tourniquet (Rummel tourniquet, not shown) is placed loosely around the common carotid artery. A filter, collapsed by a sheath, is inserted downstream the plaque through an incision made on the internal carotid artery and expanded by removing the sheath. A Bulldog clamp (not shown) is then secured on the internal carotid artery. Next, a DeBakey clamp (not shown) is placed on the common carotid artery proximal (upstream) of the tourniquet. The external carotid artery is secured with a Bulldog clamp (not shown). This order of vessel clamping is significant because the clamp on the internal carotid artery is effective to catch any embolic debris dislodged by the DeBakey clamp placed on the common carotid artery. If the patient requires a shunt, shunt 20 as depicted in FIG. 9A is inserted over sheath 60 and advanced downstream of the lesion. Shunt 20 has lumen 30 that communicates with lumen 70, and is adapted for perfusion of blood. Shunt 20 also includes proximal port 71 that is releasably attachable to a shunt (not shown) inserted upstream the lesion. Sheath 60 is then removed to free-up lumen 30 for perfusion of blood.

In an alternative approach, with the clamp in place, the surgeon makes a longitudinal incision in the artery which contains plaque material. The collapsed filter and sheath as described in FIG. 9A are inserted through the arteriotomy and advanced downstream the atheromatous lesion in the internal carotid artery. The sheath is then withdrawn to expand the filter to cover a substantial portion of the cross-sectional area of the artery. The construction and use of an expansion frame, associated filter mesh 42, and control mechanism 43 have been thoroughly discussed in earlier applications including Barbut et al., U.S. application Ser. No. 08/553,137, filed Nov. 7, 1995, now abandoned; Barbut et al., U.S. application Ser. No. 08/580,223, filed Dec. 28, 1995, now abandoned; Barbut et al., U.S. application Ser. No. 08/584,759, filed Jan. 9, 1996, now abandoned; Barbut et al., U.S. Pat. No. 5,769,816; Barbut et al., U.S. application Ser. No. 08/645,762, filed May 14, 1996; Barbut et al., U.S. Pat. No. 5,662,671, and Tsugita et al., U.S. Pat. No. 6,042,598; and the contents of each of these prior applications are incorporated herein by reference in their entirety. It will be understood that the design and use of a filter mesh, associated expansion frame, and control mechanism as discussed in these patents and applications is fully applicable to the use of such filter and expansion frame on a guidewire or arterial catheter system as disclosed herein.

If the patient requires a shunt, the distal region of shunt 20 is inserted over sheath 60 and gripped with forceps. The Bulldog clamp that secures the internal carotid artery is loosened to allow back-bleeding while shunt 20 is advanced distally into the internal carotid artery past the clamp. In this embodiment where the proximal and distal region of the shunt are releasably attached at end 71, the proximal region of the shunt is attached to end 71 and secured at its insertion site, typically in the common carotid artery, by a second forceps or other clamping means including tethering or tourniquet to prevent blood escape. When the shunt has been successfully placed in the internal carotid artery, it is secured by a Javid clamp to prevent further back-bleeding. It should be noted that during advancement of the distal opening of the shunt into the internal carotid artery, care must be taken to avoid scraping and thereby dislodging debris from the walls of the vessel. For this reason, the clamp on the internal carotid artery is loosened and allowed to back-bleed during the process so that retrograde blood flow blows the vessel walls apart so that shunt 20 can be advanced through the center. The second forceps secured to the proximal region of the shunt is released in order to vent air from the interior lumen of the shunt. Sheath 60 is removed from the shunt or retracted proximally and locked in position by sheath lock 61, thereby freeing lumen 30 for blood perfusion.

Next, the proximal opening of the shunt is advanced proximally into the common carotid artery until it abuts against the DeBakey clamp. A tourniquet is tightened and the DeBakey clamp released to allow the surgeon to slide the shunt further proximal. After purging air, the proximal region of the shunt is then connected to end 71. Blood flows from the common carotid artery to the internal carotid artery through lumen 70, lumen 30, and ports 59. Once the shunt and filter are in place and operational as depicted, it is generally desirable to evaluate shunt function using a Doppler probe. An audible flow signal will typically confirm patency. The endarterectomy procedure is then performed within the dissected region of the artery. The plaque or atheroma material typically has the consistency of a thick shell. This material is dissected and peeled out of the vessel, preferably in one or a small number of large pieces. Such a monolithic removal is preferred to breaking of the plaque into small pieces as the latter may be lost in the circulation and result in emboli. The dissected vessel is then closed by suturing both ends of the slit toward the center until a small hole remains in the common carotid artery, as described in Loftus, Carotid Endarterectomy Principles and Techniques; Quality Medical Publishing, Inc.: St. Louis, Mo., 1995. The shunt is then gripped by two clamps spaced by a short distance and removed after disconnecting its proximal and distal regions at end 71. The filter remains in the artery during shunt removal to capture emboli dislodged during shunt removal.

The clamp on the internal carotid artery is briefly loosened and allowed to back-bleed in order to purge air from the dissected region of the artery. The clamp on the external carotid artery is similarly loosened briefly to back-bleed and purge air from the affected segment of the external carotid artery. The surgeon checks for thrombi disposed within the affected segment of the vessel, and for inadvertent closure from the suture line having caught an unintended portion of the back of the vessel. Heparinized saline 65 is injected into the small opening which remains as depicted in FIG. 9B. Filter 12 is contracted to a small diameter by advancing sheath 60 distally, holding captured embolic material trapped within the mesh. The filter and sheath are then withdrawn from the artery, and removed. The last suture 66, generally comprising free ends of 6-0 prolene, is tied to completely close the incision in the dissected region of the artery. The clamp on the external carotid artery is removed, and the clamp on the common carotid artery is removed. After a delay of 10 seconds, the clamp on the internal carotid artery is removed. This sequence ensures that any inadvertent debris or air is flushed to the external carotid artery rather than the internal carotid artery and the patient thereby avoids neurologic harm. Alternatively, the filter can be left in place until blood flow is reestablished as described above.

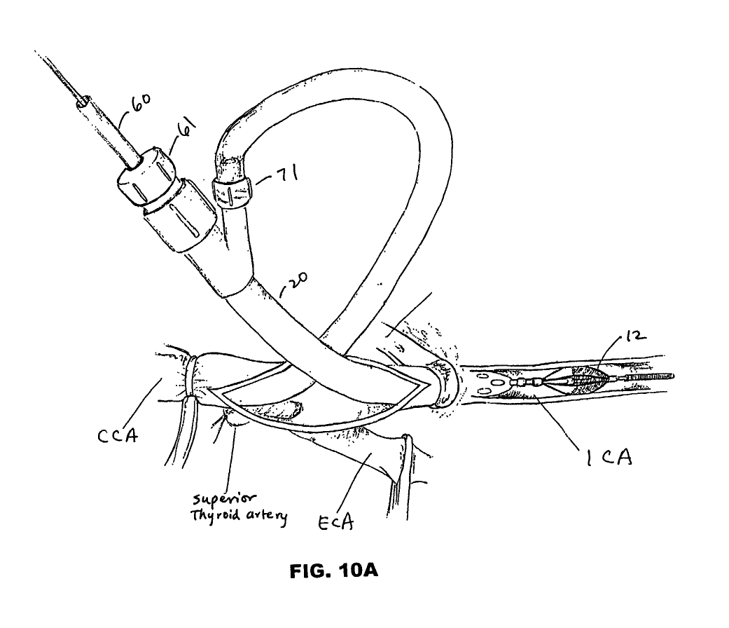

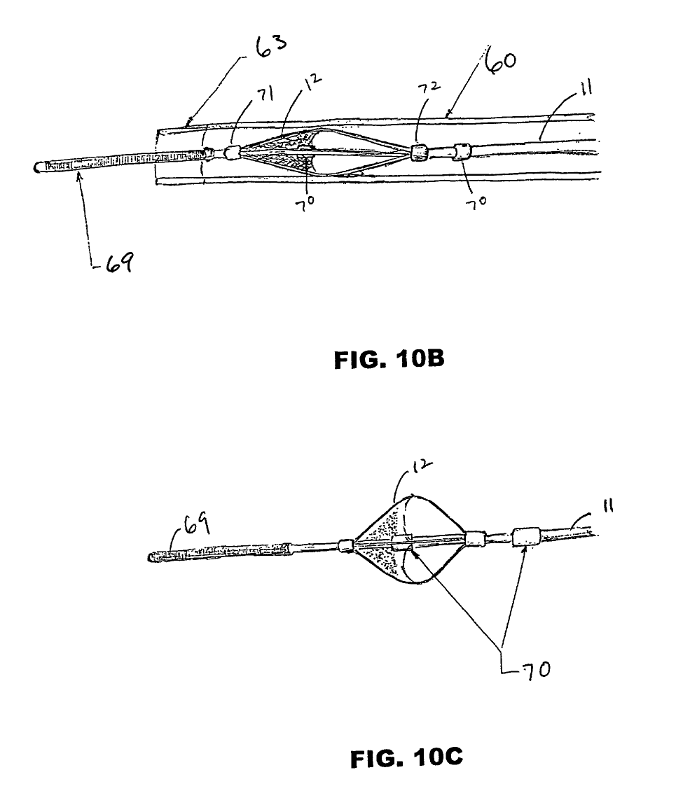

According to FIG. 10A, the detachable shunt described in FIG. 9A is inserted over another embodiment of filter 12 deployed in the internal carotid artery. Filter 12, slideably mounted on wire 11, is inserted in a collapsed state by advancing sheath 60 over the filter as depicted in FIG. 10B. Alternatively, filter 12 may be fixed to wire 11 at proximal end 72 of the expansion frame, while the distal end 71 slides over wire 11. This design allows sheath 60 to capture and close filter 12 when advanced distally. The sheath is used as a guidewire for insertion of a shunt and can be clamped over by a DeBakey or Javid clamp, thereby providing a safer alternative than clamping over the wire. Filter 12 and sheath 60 also include, respectively, atraumatic tip 69 and 63 at their distal ends to minimize trauma to the vessel wall during their insertion. Filter 12 is expanded by removing sheath 60 as depicted in FIG. 10C. The filter device also includes stoppers 70 mounted on wire 11. The stoppers allow restricted movement of the filter on wire 11 during endarterectomy, thereby stabilizing the filter on the vessel.

The introducer sheath 60 will typically have an external diameter of 5-12 French, more preferably 6-8 French. With reference to the filter device, the diameter at the distal end will typically be 1-3 mm, more preferably 1.5-2.5 mm. The filter is generally activated from the proximal end and is deployed from within a small sheath or on the outside of a guidewire or small tube. The length of the filter device is generally 20-40 cm and the deployed diameter of filter mesh 42 will typically be 2 mm or larger, more preferably 4 mm or larger, more preferably 6 mm or larger, more preferably 8 mm or larger, more preferably 10 mm or larger, and generally will be 2-10 mm. The foregoing ranges are set forth solely for the purpose of illustrating typical device dimensions. The actual dimensions of a device constructed according to the principles of the present disclosure may obviously vary outside of the listed ranges without departing from the basic principles disclosed herein.

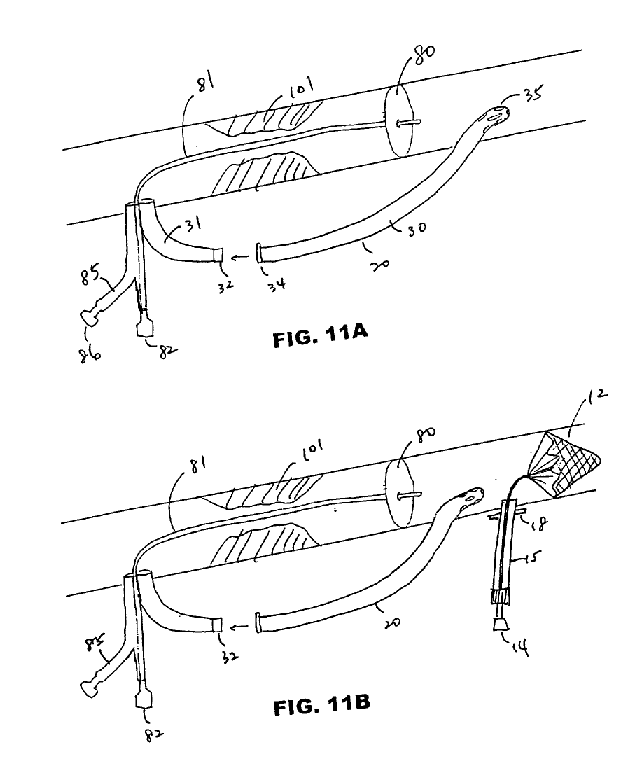

In another embodiment, a balloon occluder is used to isolate blood flow downstream an atheromatous lesion instead of using a clamp as depicted in FIG. 11A Expandable balloon 80, e.g., a toroidal balloon, is mounted on the distal region of elongate tubular member 81 and communicates proximally with inflation port 82. Lumen 85 communicates with port 86, adapted for attachment to an irrigation device, e.g., a syringe, and/or a vacuum. Distal end 32 of perfusion lumen 31 is reversibly attached to proximal end 34 of shunt 20. Blood is delivered from lumen 31 upstream lesion 101, through lumen 30 and perfusion ports 35 of shunt 20, and downstream lesion 101 and balloon occluder 80. Using the balloon occluder or an expandable occlusion membrane minimizes trauma to the vessel wall, e.g., hematoma, dissection, and plaque rupture commonly associated with using a vascular clamp. Prior to insertion of the occluder and shunt of FIG. 11A, the filter device described in FIG. 1B is inserted and deployed downstream occluder 80 and shunt 20 to protect against distal embolization as shown in FIG. 11 B. After endarterectomy, patency of the re-established vascular lumen can be assessed by infusing radio-opaque material through lumen 85 under fluoroscopy.

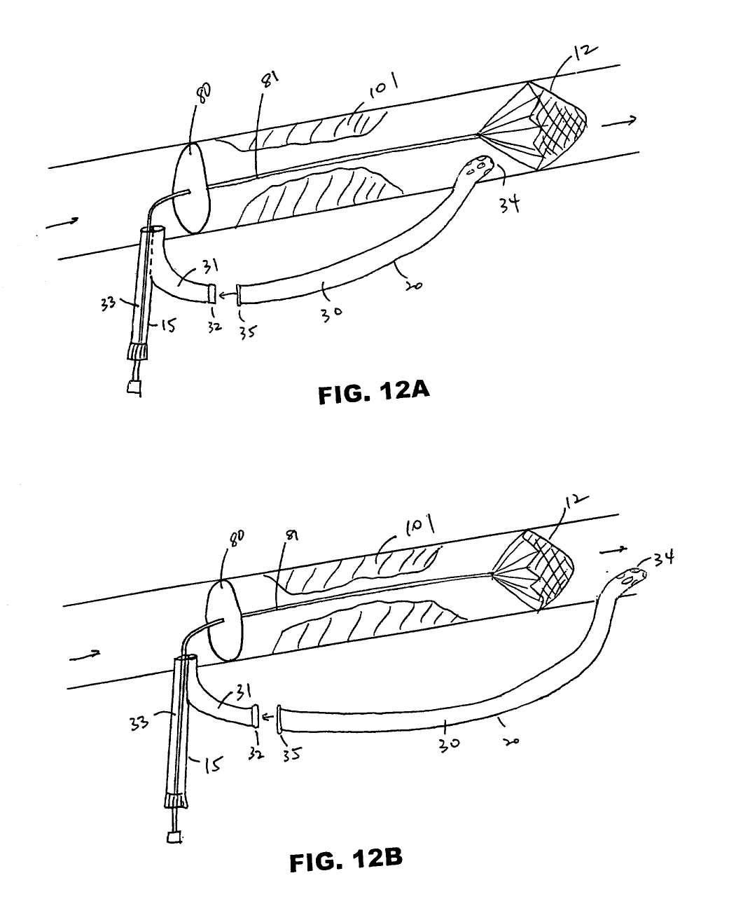

FIG. 12A depicts expandable balloon 80 mounted on a distal region of catheter 81, deployed upstream atheromatous lesion 101 to isolate blood flow. Catheter 81 is insertable through lumen 33 of the introducer. Expandable filter 12 is also mounted on catheter 81. Lumen 33 may or may not communicate with lumen 31. The proximal end of lumen 33 includes a hemostatic valve to prevent blood loss. Lumen 31 is adapted for perfusion of blood. In use, the introducer is inserted through an incision upstream lesion 101. Filter 12 is inserted in a collapsed state and deployed downstream lesion 101. Balloon 80 is inflated to occlude proximal blood flow by infusing air or fluid, such as saline. If a shunt is required, shunt 20 is attached to lumen 31 through end 32. The distal end of the shunt, which includes a plurality of perfusion ports, is inserted through an incision downstream lesion 101 and proximal to filter 12. Alternatively, the distal end of the shunt can be inserted through an arteriotomy. In an alternative method, the distal end of shunt 20 is inserted downstream lesion 101 and filter 12 as depicted in FIG. 12B.

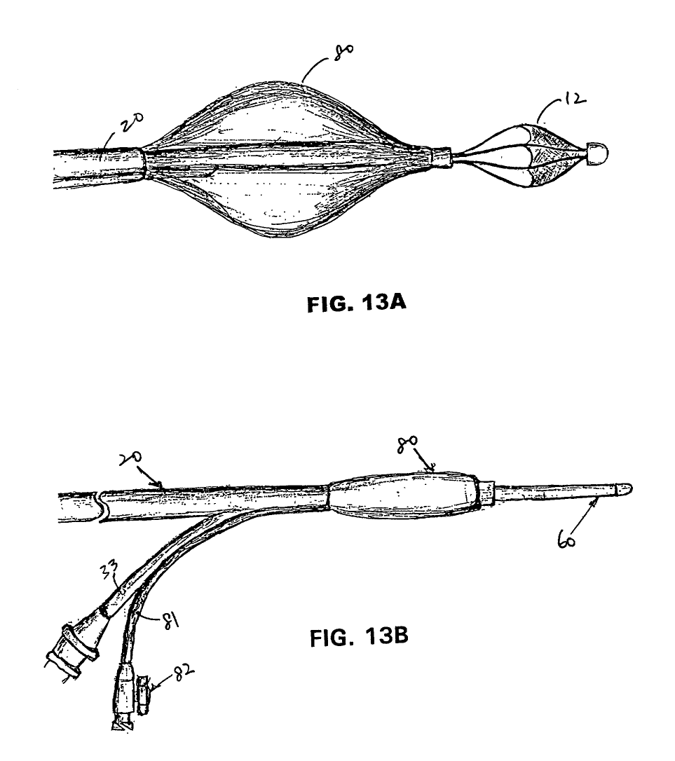

In another embodiment, the shunt is secured to the vessel walls using a balloon occluder mounted on a distal region of the shunt as depicted in FIGS. 13A and 13B. Expandable balloon 80 is disposed circumferentially around the tubing of shunt 20. Balloon 80 is in fluid communication with inflation lumen 81 and inflation port 82. The shunt also includes lumen 33 adapted for insertion of filter 12 and sheath 60. In use, filter 12 is introduced in a collapsed state, covered by sheath 60, through lumen 33. The filter is deployed downstream an atheromatous lesion by withdrawing the sheath proximally. The distal end of shunt 20 is then inserted over the sheath and the wire of the filter. The distal end of the shunt is then positioned as described above, while occluder 80 is in a deflated state. Back-bleeding through the shunt occurs from distal ports 35 in order to purge air from within the shunt. After the shunt is purged, saline, or other biotolerable fluid, is injected through port 82 until occluder 80 enlarges into contact with the inner diameter of the vessel, thereby sealing the vessel from blood flow and securing the shunt to the vessel. A cuff or C-clamp may be fitted about the vessel to prevent hyperexpansion, minimize internal slippage of the balloon occluder, and provide a tight seal within the vessel. After the endarterectomy procedure, saline is withdrawn to deflate occluder 80 before the shunt is removed from the vessel. Sheath 60 is then advanced over filter 12 to collapse the filter.

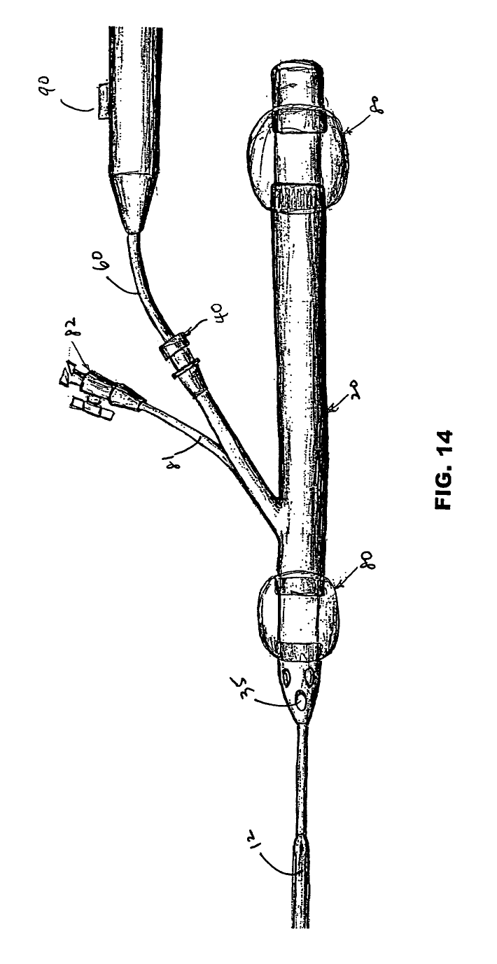

In another embodiment, the shunt is secured to the vessel walls using two balloon occluders as depicted in FIG. 14. Balloon occluders 80 are disposed circumferentially around the proximal and distal regions of the shunt. Occluders 80 communicate with an inflation lumen and proximally with inflation port 82. The distal end of shunt 20 includes a plurality of perfusion ports 35, which facilitate laminar flow. Filter 12, collapsed within sheath 60, is insertable through lumen 33, which includes hemostatic valve 40 at its proximal end to prevent blood loss. Sheath 60 is attached to filter deploying mechanism 90 at its proximal end. In use, after sheath 60 and collapsed filter 12 are inserted into the internal carotid artery, filter 12 is deployed by operating mechanism 90, thereby withdrawing the sheath proximally. Shunt 20, having the occluder in a deflated state, is then inserted over the sheath and the wire of the filter to position within the region of interest. Balloon occluders 80 are expanded to seal the vessel from blood flow. After the endarterectomy procedure, occluders 80 are deflated, shunt 20 is removed from the vessel, and filter 12 is collapsed by sheath 60 and removed from the vessel.

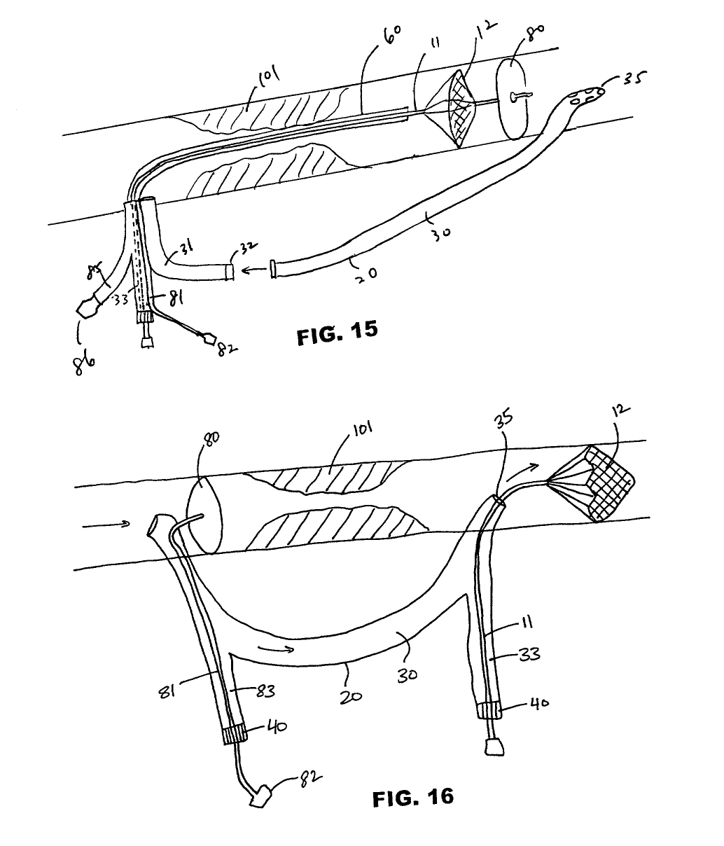

FIG. 15 depicts another embodiment of the introducer which includes lumen 31 for perfusion of blood, lumen 85 for irrigation and aspiration, lumen 33 for insertion of sheath 60 and filter 12. Prior to insertion of the introducer, filter 12 is collapsed and covered by sheath 60 and balloon 80 is deflated. After inserting the introducer upstream lesion 101, filter 12 is expanded by withdrawing sheath 60 proximally, and balloon 80 is expanded by infusing saline through port 82 and lumen 81. If a shunt is required, shunt 20 communicates with lumen 31 by attaching to end 32. The distal end of the shunt is inserted through an incision downstream lesion 101 distal to filter 12 and balloon 80.

FIG. 16 depicts another embodiment of the shunt which includes lumen 83 and lumen 33. Lumen 83 is adapted for insertion of balloon occluder 80 which communicates with inflation lumen 81. Lumen 33 is adapted for insertion of filter 12 mounted on wire 11. Lumen 30 of shunt 20 communicates with lumens 83 and 33. The proximal ends of lumens 83 and 33 include hemostatic valves 40 to prevent blood loss. In use, the distal end of shunt 20, carrying collapsed filter 12 in lumen 33, is inserted downstream lesion 101. The filter is expanded. The shunt and filter are secured by a clamp. The proximal end of shunt 20, having balloon occluder 80 in a deflated state, is inserted upstream lesion 101. The shunt is allowed to back-bleed from distal ports 35 in order to purge air from within the shunt. After the shunt is purged, occluder 80 is expanded to occlude proximal blood flow. After endarterectomy is performed according to the procedure described above, occluder 80 is deflated, shunt 20 is removed, and then filter 12 is collapsed and removed.

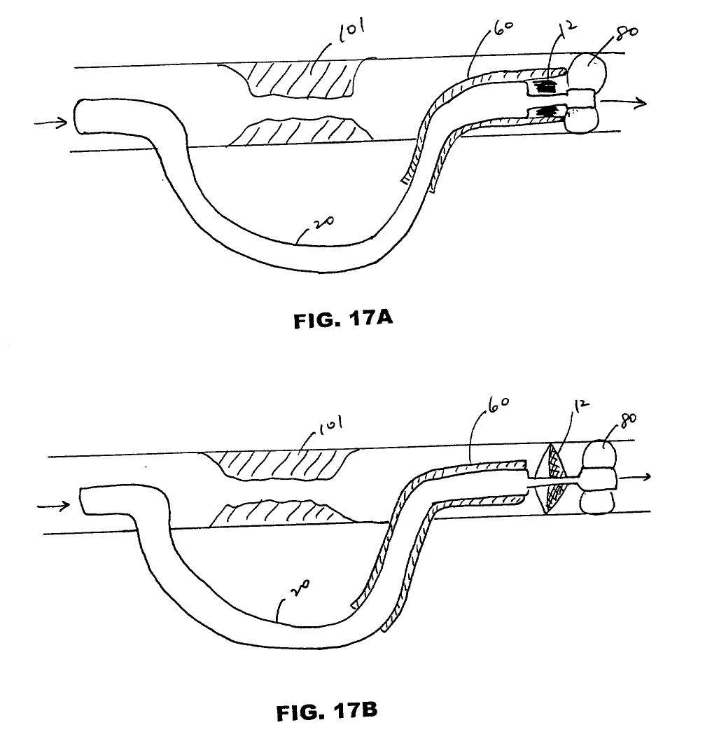

In another embodiment, filter 12 and balloon occluder 80 are mounted on a distal region of shunt 20 as depicted in FIGS. 17A and 17B. Sheath 60 is slideably disposed about the distal region of the shunt to cover the filter, and is retractable to release the filter. The shunt may also contain a recess shaped to receive filter 12 when the filter is closed. Balloon occluder 80 is mounted distal filter 12. In use, the distal end of the shunt, having deflated balloon occluder 80 and collapsed filter covered by sheath 60, is inserted through an incision downstream atheromatous lesion 101. Alternatively, the shunt is inserted through an opening in the vessel after arteriotomy. Sheath 60 is pulled back to expand filter 12 as depicted in FIG. 17B. Balloon occluder 80 is then inflated to occlude the lumen of the vessel and secure the distal end of the shunt. The proximal end of the shunt is inserted upstream lesion 101 and secured by a clamp. After endarterectomy, balloon occluder 80 is deflated and sheath 60 is advanced distally to collapse filter 12. The filter, with captured embolic material, and the shunt are then removed from the vessel. The incisions are closed with sutures.

It will be understood that filtration is an important aspect of the endarterectomy shunt and methods disclosed herein. To filter blood effectively, i.e., to capture embolic material, without unduly disrupting blood flow, the mesh must have the appropriate physical characteristics, including area (AM), thread diameter (DT), and pore size (Sp). The characteristics of the emboli and the filter for use in the carotid artery have been thoroughly discussed in et. al., U.S. Pat. No. 5,876,367, Mar. 2, 1999, incorporated herein by reference in it entirety. Other suitable filter materials include parylene, PET, polyurethane, nitinol, plastic membrane, or metal foil.

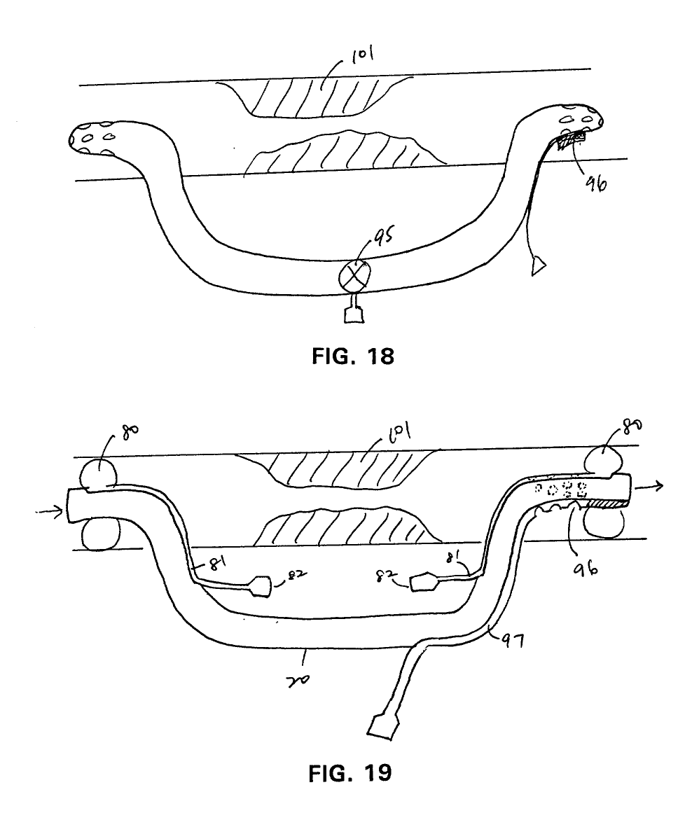

Another embodiment of the shunt which includes a valve for controlling blood flow is depicted in FIG. 18. Valve 95 can be included in the proximal, distal, or mid-section of the shunt. Manometer 96 is mounted on the distal end of the shunt for measuring blood pressure downstream lesion 101. Since cerebral tissue generally can not tolerate extreme pressure fluctuation, cerebral perfusion can be regulated by adjusting valve 95 according to pressure readings obtained by manometer 90.

FIG. 19 depicts another embodiment of the shunt, which includes balloon occluders 80 disposed about proximal and distal ends of shunt 20. Each occluder communicates with an inflation lumen 81 and port 82, such that each occluder can be independently inflated and deflated. The shunt also includes infusion/aspiration lumen 97, which communicates distally with a plurality of ports 96.

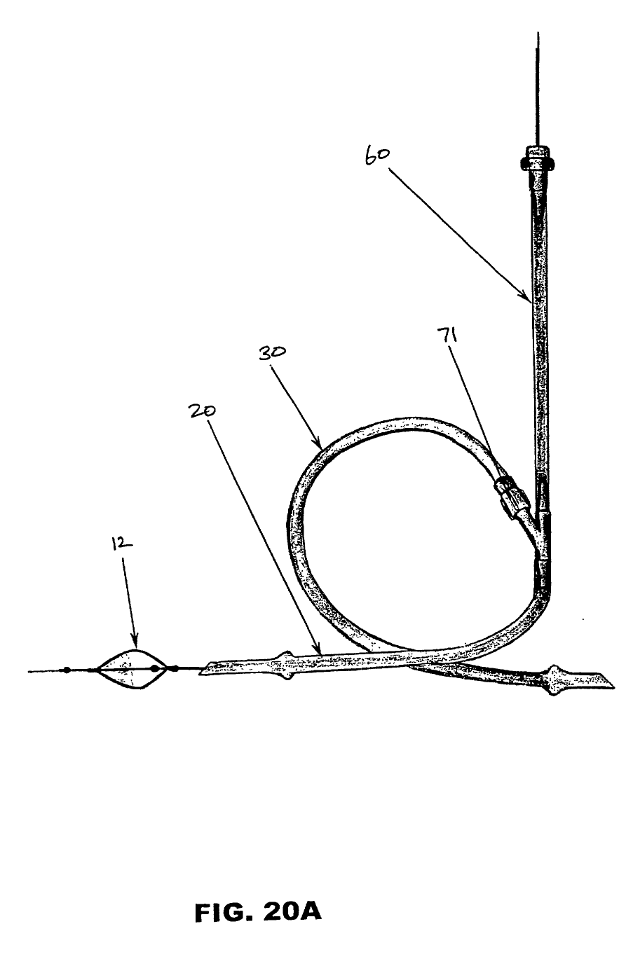

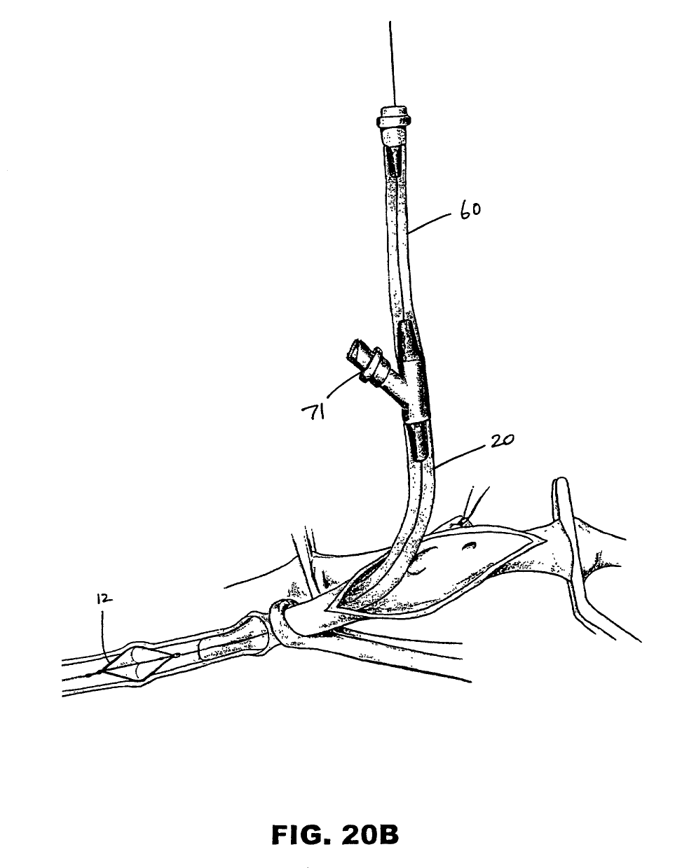

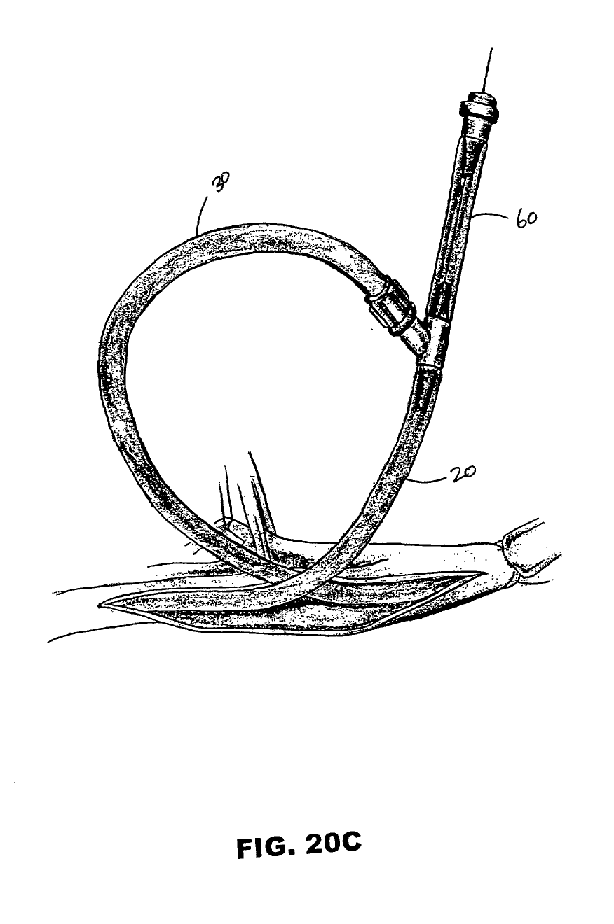

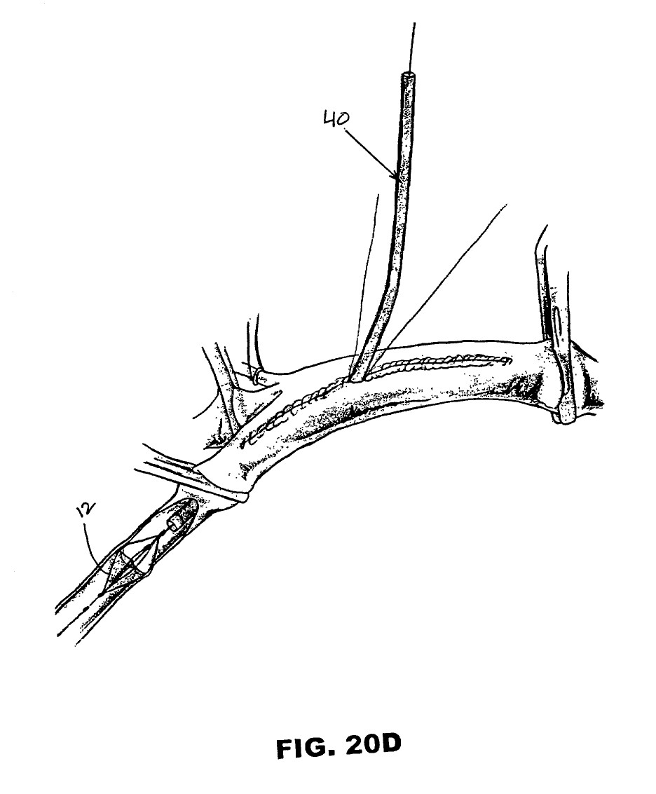

In another embodiment as shown in FIG. 20A, the shunt consists of distal portion 20 with filter deployment port 60 and optional proximal portion 30, which can be connected to the distal portion using the Y-connector 71. During the procedure, the distal portion of the shunt, without the proximal portion attached, is inserted into the distal end of the arteriotomy. Filter 12 is then deployed as shown in FIG. 20B. In FIG. 20B, the distal portion of the shunt is a separate stick filter insertion port with Y-connector for the optional proximal portion of the shunt.

In use, the shunt is installed as shown in FIG. 20C. After the surgeon has completed the removal of the plaque and cleaning of the arterial surface, normal closing procedures can begin. The closing procedure, either with or without a patch, will be similar to those procedures above using a shunt. The arteriotomy is closed as much as possible before removing the proximal end of the shunt. The proximal end of the shunt is removed from the arteriotomy and disconnected from the Y-connector. Retrieval sheath 40 is inserted over the filter wire as shown in FIG. 20D. The distal portion of the shunt is removed over the retrieval sheath, leaving filter 12 deployed. The suture is tightened around the retrieval sheath. Blood flow is restored, and the surgeon performs any necessary imaging. The filter is retracted into retrieval sheath 40. The retrieval sheath and filter are removed. The arteriotomy is closed completely by suturing.

The length of the shunt will generally be between 5 and 20 centimeters, more preferably approximately between 10 and 15 centimeters. The inner diameter of the shunt adapted for arterial perfusion will generally be between 0.5 and 1.5 centimeters, preferably approximately 1.0 centimeters. The length of the wire and sheath adapted for insertion of the filter device will generally be between 5.0 and 30.0 centimeters, preferably approximately 15 centimeters. The inner diameter of the sheath will generally be between 0.2 and 1.0 centimeters, preferably approximately 0.4 centimeters. The foregoing ranges are set forth solely for the purpose of illustrating typical device dimensions. The actual dimensions of a device constructed according to the principles of the present invention may obviously vary outside of the listed ranges without departing from those basic principles.

Although the foregoing invention has, for purposes of clarity of understanding, been described in some detail by way of illustration and example, it will be obvious that certain changes and modifications may be practiced which will still fall within the scope of the appended claims. For example, the devices and methods of each embodiment can be combined with or used in any of the other embodiments.

What is claimed is:

1. A method for open surgical endarterectomy, comprising the steps of:
providing an elongate member having a proximal end, a distal end, and an expandable filter at the distal end;
inserting the distal end of the elongate member into the internal carotid artery downstream of a lesion;
expanding the filter;
advancing a tubular member having proximal and distal ends over the elongate member to place the distal end of the tubular member within the internal carotid artery;
inserting the proximal end of the tubular member into the common carotid artery upstream of the lesion; and
flowing blood from the common carotid artery through a lumen of the tubular member into the internal carotid artery.

2. The method of claim 1, wherein the elongate member comprises a wire.

3. The method of claim 1, wherein the filter is fixedly mounted on the elongate member.

4. The method of claim 1, wherein the filter is slideably mounted on the elongate member.

5. The method of claim 1, wherein the elongate member further comprises a sheath covering the filter, and wherein the method further comprises the step of withdrawing the sheath to release the filter.

6. The method of claim 1, wherein the filter comprises a plurality of flexible struts, each strut bonded to the elongate member at a proximal end, and each strut having a distal end slideably mounted on the elongate member.

7. The method of claim 1, further comprising the steps of occluding the common carotid artery upstream of the lesion and occluding the internal carotid artery downstream of the lesion.

8. The method of claim 7, wherein the common carotid artery and internal carotid artery are occluded by clamping.

9. The method of claim 1, further comprising the step of back-bleeding the tubular member to purge air.

10. The method of claim 7, further comprising the step of making an arteriotomy to access the lesion.

11. The method of claim 10, further comprising the step of removing the lesion by endarterectomy.

12. The method of claim 10, further comprising the step of suturing to close the arteriotomy.

13. The method of claim 12, further comprising the steps of removing occlusion from the common carotid and internal carotid arteries.

14. The method of claim 12, further comprising the step of removing the proximal and distal ends of the tubular member while maintaining the filter in the internal carotid artery.

15. The method of claim 12, further comprising the steps of collapsing and removing the filter.

16. The method of claim 5, further comprising the steps of advancing the sheath distally over the elongate member to cover the filter, and removing the elongate member and sheath from the internal carotid artery.

17. The method of claim 10, further comprising the step of flushing the lesion with saline.

18. The method of claim 7, further comprising the step of occluding the external carotid artery.

19. The method of claim 1, further comprising the step of making an incision on the internal carotid artery.

20. The method of claim 10, further comprising the step of applying a patch graft to close the arteriotomy.

21. The method of claim 12, further comprising the step of removing the filter while maintaining the tubular member in the internal carotid artery.

22. The method of claim 1, wherein the tubular member has a port between the proximal and distal ends, and wherein the elongate member is passed through the port as the tubular member is advanced over the elongate member.

23. The method of claim 22, wherein the port includes a hemostatic valve.

24. The method of claim 18, further comprising the step of measuring the blood pressure in the internal carotid artery before occluding the common and the external carotid arteries.

25. A method for open surgical endarterectomy, comprising the steps of:
providing an elongate member having a proximal end, a distal end, and an expandable filter at the distal end;
inserting the distal end of the elongate member into the internal carotid artery downstream of a lesion;
expanding the filter;
advancing a first tubular member having proximal and distal ends over the elongate member to place the distal end of the first tubular member within the internal carotid artery;
inserting a proximal end of a second tubular member into the common carotid artery upstream of the lesion;
joining the distal end of the second tubular member to the proximal end of the first tubular member; and
flowing blood from the common carotid artery through a lumen of the tubular member into the internal carotid artery.

26. The method of claim 25, wherein the elongate member comprises a wire.

27. The method of claim 25, wherein the filter is fixedly mounted on the elongate member.

28. The method of claim 25, wherein the filter is slideably mounted on the elongate member.

29. The method of claim 25, wherein the elongate member further comprises a sheath covering the filter, and wherein the method further comprises the step of withdrawing the sheath to release the filter.

30. The method of claim 25, wherein the filter comprises a plurality of flexible struts, each strut bonded to the elongate member at a proximal end, and each strut having a distal end slideably mounted on the elongate member.

31. The method of claim 25, further comprising the steps of occluding the common carotid artery upstream of the lesion and occluding the internal carotid artery downstream of the lesion.

32. The method of claim 31, wherein the common carotid artery and internal carotid artery are occluded by clamping.

33. The method of claim 25, further comprising the step of back-bleeding the tubular member to purge air.

34. The method of claim 31, further comprising the step of making an arteriotomy to access the lesion.

35. The method of claim 34, further comprising the step of removing the lesion by endarterectomy.

36. The method of claim 34, further comprising the step of suturing to close the arteriotomy.

37. The method of claim 36, further comprising the steps of removing occlusion from the common carotid and internal carotid arteries.

38. The method of claim 36, further comprising the step of removing the proximal and distal ends of the tubular member while maintaining the filter in the internal carotid artery.

39. The method of claim 36, further comprising the steps of collapsing and removing the filter.

40. The method of claim 29, further comprising the steps of advancing the sheath distally over the elongate member to cover the filter, and removing the elongate member and sheath from the internal carotid artery.

41. The method of claim 34, further comprising the step of flushing the lesion with saline.

42. The method of claim 31, further comprising the step of occluding the external carotid artery.

43. The method of claim 25, further comprising the step of making an incision on the internal carotid artery.

44. The method of claim 34, further comprising the step of applying a patch graft to close the arteriotomy.

45. The method of claim 36, further comprising the step of removing the filter while maintaining the tubular member in the internal carotid artery.

46. The method of claim 25, wherein the tubular member has a port between the proximal and distal ends, and wherein the elongate member is passed through the port as the tubular member is advanced over the elongate member.

47. The method of claim 46, wherein the port includes a hemostatic valve.

48. A method for open surgical endarterectomy, comprising the steps of:
providing an elongate member having a proximal end, a distal end, an expandable filter at the distal end, and a slideable sheath covering the filter;
making an incision on the internal carotid artery downstream of a lesion;
inserting the distal end of the elongate member through the incision;
withdrawing the sheath from the filter;
expanding the filter downstream of the lesion;
occluding the external carotid artery, common carotid artery, and internal carotid artery to isolate a region of the internal carotid artery with the lesion;
performing endarterectomy to remove the lesion; and
removing occlusion from the external carotid artery, common carotid artery, and internal carotid artery, wherein embolic material is captured by the filter.

49. The method of claim 48, further comprising the steps of flushing and aspirating embolic material from the region.

50. The method of claim 48, wherein the endarterectomy is performed through an arteriotomy on the internal carotid artery.

51. The method of claim 50, further comprising the step of suturing to close the arteriotomy.

52. The method of claim 50, further comprising the step of applying a patch to close the arteriotomy.

53. The method of claim 48, wherein the endarterectomy is performed through an arteriotomy on the internal carotid artery.

54. The method of claim 48, wherein the external carotid, common carotid, and internal carotid arteries are occluded by clamping.

55. The method of claim 48, wherein the elongate member comprises a wire.

56. The method of claim 48, wherein the filter is fixedly mounted on the elongate member.

57. The method of claim 48, wherein the filter is slideably mounted on the elongate member.

58. The method of claim 48, wherein the filter comprises a sheath covering the filter, and wherein the method further comprises the step of withdrawing the sheath to release the filter.

59. The method of claim 48, further comprising the steps of collapsing and removing the filter.

60. The method of claim 58, further comprising the steps of advancing the sheath distally over the elongate member to cover the filter, and removing the elongate member and sheath from the internal carotid artery.

61. A medical device for open surgical endarterectomy, comprising:
a first tubular member having a proximal end, a distal end, and a lumen therebetween communicating with a distal port, a first proximal port, and a second proximal port;
a hemostatic valve mounted in the second proximal port;
a second tubular member having a proximal end, a distal end, and a lumen therebetween, the distal end of the second tubular member adapted for releasable attachment to the first proximal port of the first tubular member; and
an elongate member inserted through the hemostatic valve and the second proximal port, the elongate member having a proximal end, a distal end, and an expandable filter at the distal end.

62. The medical device of claim 61, wherein the elongate member further comprises a slideable sheath covering the filter.

63. The medical device of claim 61, wherein the distal end of the first tubular member includes a plurality of ports.

64. The medical device of claim 61, wherein the proximal end of the second tubular member includes a plurality of ports.

65. The medical device of claim 61, wherein an expandable balloon is mounted on the distal end of the first tubular member.

66. The medical device of claim 61, wherein an expandable balloon is mounted on the proximal end of the second tubular member.

67. The medical device of claim 66, wherein the balloon is a toroidal balloon.

68. The medical device of claim 66, wherein the balloon communicates with an inflation lumen.

69. The medical device of claim 61, wherein the first tubular member further comprises a valve for regulating blood flow.

70. The medical device of claim 61, wherein the second tubular member further comprises a valve for regulating blood flow.

71. The medical device of claim 61, wherein the first tubular member further comprises a lumen adapted for irrigation and aspiration.

72. The medical device of claim 71, wherein the lumen communicates with a plurality of aspiration ports.

73. The medical device of claim 61, wherein the distal end of the first tubular member includes a suture flange.

74. The medical device of claim 61, wherein the proximal end of the second tubular member includes a suture flange.

75. The medical device of claim 61, wherein the distal end of the first tubular member includes a manometer.

76. The medical device of claim 61, wherein the distal end of the filter includes an atraumatic tip.

77. A shunt, comprising:
an elongate tubular member having a proximal end, a distal end, and a lumen therebetween;
an expandable filter mounted on the distal end of the shunt; and
a sheath slideably disposed about the distal end of the elongate tubular member and covering the filter,
wherein the sheath is retractable to release the filter.

78. The shunt of claim 77, further comprising a balloon mounted on the elongate tubular member distal the filter.

79. The shunt of claim 77, wherein the elongate tubular member contains a recess shaped to receive the filter when the filter is closed.

80. The shunt of claim 78, wherein the balloon communicates with an inflation lumen.

81. The shunt of claim 77, further comprising a balloon mounted on the proximal end of the elongate tubular member.

82. The shunt of claim 78, wherein the shunt further includes a plurality of aspiration ports proximal the balloon.

83. The medical device of claim 77, wherein the distal end of the filter includes a manometer.

84. The method of claim 48, wherein endarterectomy is performed through the incision on the internal carotid artery downstream of a lesion through which the distal end of the elongate member is inserted.

85. The method of claim 48, further comprising the step of making a second incision on the internal carotid artery, wherein endarterectomy is performed through the second incision on the internal carotid artery.
